(19) United States
(12) Patent Application Publication
(10) Pub. No.: US 2025/0034531 A1
(43) Pub. Date: Jan. 30, 2025

(54) METHOD FOR PRODUCING STEM CELLS

(71) Applicants: Koji TANABE, Palo Alto, CA (US); I Peace, Inc., Palo Alto, CA (US)

(72) Inventors: Koji TANABE, Palo Alto, CA (US); Kenta SUTO, Kyoto-shi (JP)

(73) Assignees: Koji TANABE, Palo Alto, CA (US); I Peace, Inc., Palo Alto, CA (US)

(21) Appl. No.: 18/696,908

(22) PCT Filed: Sep. 15, 2022
(86) PCT No.: PCT/JP2022/034609

§ 371 Date: Mar. 28, 2024

Related U.S. Application Data
(60) Provisional application No. 63/250,118, filed on Sep. 29, 2021.

(51) Int. Cl. C12N 5/074

(52) U.S. Cl. CPC C12N 5/0696; C12N 2500/42; C12N 2501/2302; C12N 2510/00

(57) ABSTRACT

According to the present disclosure, there is provided a method for producing stem cells, which includes applying a phosphate to blood cells and inducing stem cells from the blood cells. The phosphate may be an intermediate product or a final product in a non-mevalonate pathway. The phosphate may be (E)-4-hydroxy-3-methyl-but-2-enyl pyrophosphate.

### TECHNICAL FIELD

The present invention relates to a cell technique and relates to a method for producing stem cells.

### BACKGROUND ART

T cells derived from hematopoietic stem cells play an important role in immunity. T cells express, on the surface thereof, T cell receptors (TCRs) which are rich in variety. The diversity of TCRs is brought about by V(D)J rearrangement. The VJ rearrangement is illustrated in FIG. 1.

In response to interleukin-7 (IL-7) and a c-kit ligand (KL), which are strongly expressed in the subcapsular cortex region of the thymus, double negative 1 (DN1) cells, which are progenitor cells of the T cells, proliferate and express CD25 (interleukin-2 receptor a chain), thereby becoming DN2 cells. DN2 cells gradually decrease the expression of CD44 to become DN3 cells.

The TCR includes a variable (V) region and a constant (C) region. The amino acid sequence of the V region is rich in variety and forms a binding site with an antigen. The V region gene is present in the germline DNA in a state of being divided into a V gene, a D (diversity) gene, and a J (joining) gene. In the process of differentiating into T cells, these genes undergo rearrangement, thereby being connected to be V-(D)-J on the chromosome and being an expressive gene. It is noted that the J gene includes a JP1 gene, a JP gene, a J1 gene, a JP2 gene, and a J2 gene.

The DN3 cells in which the V(D)J rearrangement has occurred at loci of the TCRβ gene and the TCRγ gene become TCRαβ-type T cells or TCRγδ-type T cells. In TCRαβ-type T cells, the TCR consists of an α chain and a β chain. In TCRγδ-type T cells, the TCR consists of a γ chain and a δ chain. The branching determination of whether to become a TCRαβ-type T cell or a TCRγδ-type T cell depends on the regulation in silencer regions which are present at the TCRγ gene locus and the TCRα gene locus, respectively.

Although the number of TCRγδ-type T cells is smaller than that of TCRαβ-type T cells, the TCRγδ-type T cells occupy the majority of the intraepithelial lymphocytes in the intestinal mucosa. TCRγδ-type T cells sense various stresses that damage cells and induce an immune response. It has been reported that TCRγδ-type T cells sense changes in properties associated with canceration of cells in addition to stress from outside the body, such as bacterial infection or viral infection.

TCRγδ-type T cells recognize, as an antigen, an intermediate product in the metabolism of mevalonic acid in the cholesterol synthesis pathway of the antigen presenting cell (APC) or isopentenyl pyrophosphate (IPP) and then proliferate or are activated. Therefore, cancer immunotherapy is carried out, where TCRγδ-type T cells of a patient are activated in vitro and returned to the body of the patient. However, TCRγδ-type T cells are present in only 1% to 5% of the peripheral blood, and thus there is a problem that sufficient TCRγδ-type T cells cannot be obtained even in a case where blood collection has been carried out.

Therefore, it has been proposed to initialize blood cells stimulated with zoledronic acid and then induce iPS cells having a γδ-TCR gene that has undergone the rearrangement (see, for example, Patent Document 1). However, in the iPS cells induced by this method, it has been reported that a γδ-TCR gene having a J1/J2 gene has not undergone rearrangement (for example, see Non-Patent Document 1).

### CITATION LIST

### Patent Document

Patent Document 1: International Publication No. 2018/143243

### Non-Patent Document

Non-Patent Document 1: DAISUKE WATANABE et al., “The Generation of Human cdT Cell-Derived Induced Pluripotent Stem Cells from Whole Peripheral Blood Mononuclear Cell Culture,” STEM CELLS TRANSLATIONAL MEDICINE, 2018; 7:34-44

### SUMMARY OF INVENTION

### Technical Problem

An object of the present invention is to provide a method for efficiently inducing a stem cell having a γδ-TCR gene that has undergone rearrangement.

### Solution to Problem

A method for producing stem cells according to an aspect of the present invention includes applying a phosphate to blood cells and inducing stem cells from the blood cells.

In the above-described method for producing stem cells, the phosphate may be an intermediate product or a final product in a non-mevalonate pathway.

In the above-described method for producing stem cells, the phosphate may be (E)-4-hydroxy-3-methyl-but-2-enyl pyrophosphate.

The above-described method for producing stem cells may further include applying an interleukin to the blood cells.

In the above-described method for producing stem cells, the interleukin may be at least one selected from the group consisting of IL-2, IL-15, and IL-23.

In the above-described method for producing stem cells, the blood cells may be monocytes.

In the above-described method for producing stem cells, the stem cell may be an iPS cell.

In the above-described method for producing stem cells, in the inducing of the stem cell from the blood cells, an inducer RNA may be introduced into the blood cells.

In the above-described method for producing stem cells, in the inducing of the stem cell from the blood cells, a Sendai virus vector may be used.

In the above-described method for producing stem cells, in the inducing of the stem cell from the blood cells, a stealth type RNA vector may be used.

In the above-described method for producing stem cells, the stem cell may contain a rearranged γδ-TCR gene.

A method for producing blood cells according to an aspect of the present invention includes preparing stem cells produced by the above-described method for producing stem cells and inducing a blood cell from the stem cells.

In the above-described method for producing blood cells, the blood cell may be a γδ-type T cell.

In the above-described method for producing blood cells, in the inducing of the blood cell from the stem cells, cell clumps of the stem cells may be seeded on feeder cells.

In the above-described method for producing blood cells, the feeder cells may be stromal cells (stroma cells). The stromal cells may be derived from the bone marrow. The stromal cell may be an OP9 cell.

### Advantageous Effects of Invention

According to the present invention, it is possible to provide a method for efficiently inducing a stem cell having a γδ-TCR gene that has undergone rearrangement.

### BRIEF DESCRIPTION OF DRAWINGS

FIG. 1 is a schematic view illustrating VJ rearrangement.

FIG. 2 is a graph showing the number of colonies of induced stem cells according to each of Example 1 and Comparative Example 1.

FIG. 3A is a photographic image of the induced stem cells according to Example 1.

FIG. 3B is a photographic image of the induced stem cells according to Example 1.

FIG. 4 shows photographic images showing results obtained by analyzing the genome of the induced stem cells according to each of Example 1 and Comparative Example 1 by PCR.

FIG. 5A is a photographic image of immunostained cells according to Example 1.

FIG. 5B is a photographic image of the immunostained cells according to Example 1.

FIG. 5C is a photographic image of the immunostained cells according to Example 1.

FIG. 5D is a photographic image of the immunostained cells according to Example 1.

FIG. 6 is a dot plot by a flow cytometer, which shows results of Example 1.

FIG. 7A is a photographic image of cells according to Example 2.

FIG. 7B is a photographic image of the cells according to Example 2.

FIG. 7C is a photographic image of the cells according to Example 2.

FIG. 8 is a dot plot by a flow cytometer, which shows results of Example 2.

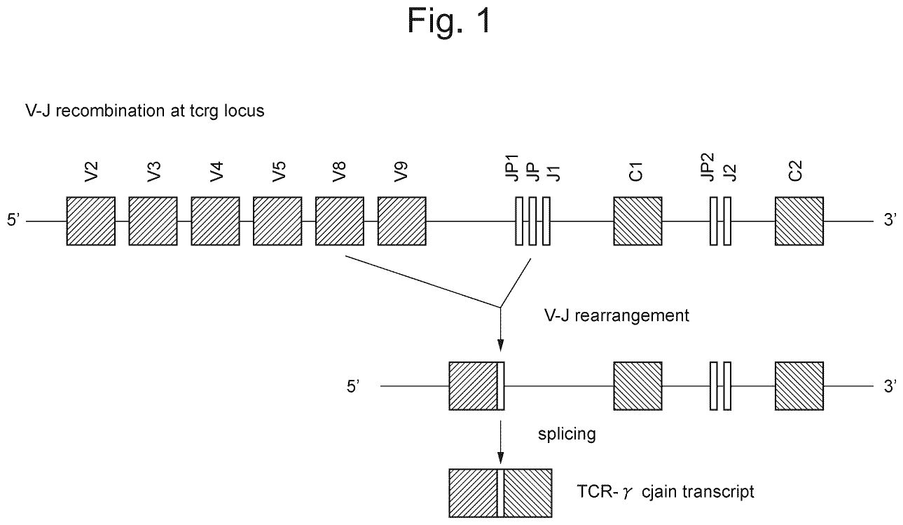

### DESCRIPTION OF EMBODIMENTS

Hereinafter, embodiments of the present invention will be described in detail. It is noted that the following embodiments are examples for embodying the technical idea of the present invention, and the technical idea of the present invention does not specify the combination of constituent members and the like to those described below. The technical idea of the present invention can be modified in various ways within the scope of the patent claims.

A method for producing stem cells according to the embodiment of the present invention includes applying a phosphate to blood cells and inducing stem cells from the blood cells. The stem cell is, for example, an induced pluripotent stem cell (iPS cell).

The blood cells may be derived from a human or may be derived from a non-human animal. The blood cells are separated from the blood. The blood is, for example, peripheral blood and umbilical cord blood; however, it is not limited thereto. The blood may be collected from an adult or may be collected from a minor. At the time of blood collection, anticoagulants such as ethylenediaminetetraacetic acid (EDTA), heparin, and a blood preserving liquid based on the biological product standards, Liquid A (ACD-A), may be used.

The blood cells are nucleated cells such as monocytes, neutrophils, eosinophils, basophils, and lymphocytes, and do not include erythrocytes, granulocytes, and thrombocytes. The blood cells may be, for example, endothelial progenitor cells, blood stem/progenitor cells, T cells, or B cells. The T cells may be, for example, αβT cells or γδT cells. The blood cells do not have to be γδT cells.

The phosphate may be an intermediate product or a final product in a non-mevalonate pathway. The non-mevalonate pathway is a synthetic pathway for isopentenyl pyrophosphate (IPP) that does not pass through mevalonic acid. Examples of the intermediate product of the non-mevalonate pathway include 1-deoxy-D-xylulose-5-phosphate (DOXP), 2-C-methyl-D-erythritol 4-phosphate (MEP), 4-diphosphositydyl-2-C-methyl erythritol (CDP-ME), 4-diphosphositydyl-2-C-methyl-D-erythritol 2-phosphate (CDP-MEP), 2-C-methyl-D-erythritol-2,4-cyclopyrrophosphate (MEcPP), and (E)-4-hydroxy-3-methyl-but-2-enyl pyrophosphate (HMB-PP).

In a case where the phosphate is applied to the blood cells, the phosphate may be added to a culture medium in which the blood cells are cultured. The concentration of the phosphate in the culture medium is, for example, 1 μmol/L or more and 50 μmol/L or less, 3 μmol/L or more and 20 μmol/L or less, or 5 μmol/L or more and 12 μmol/L or less. The blood cells may be cultured for 1 day or more, 2 days or more, or 3 days or more in the culture medium to which the phosphate has been added. The blood cells may be cultured for 14 days or less, 10 days or more, or 7 days or more in the culture medium to which the phosphate has been added.

An interleukin may be further applied to the blood cells. Examples of the interleukins include IL-2, IL-15, and IL-23.

In a case where the interleukin is applied to the blood cells, the interleukin may be added to the culture medium in which the blood cells are cultured. The concentration of the interleukin in the culture medium is, for example, 5 ng/ml or more and 100 ng/ml or less, 10 ng/ml or more and 90 ng/ml or less, or 15 ng/ml or more and 80 ng/ml or less. The blood cells may be cultured for 1 day or more, 2 days or more, or 3 days or more in the culture medium to which the interleukin has been added. The blood cells may be cultured for 14 days or less, 10 days or more, or 7 days or more in the culture medium to which the interleukin has been added.

The interleukin may be applied after the phosphate has been applied to the blood cells. The phosphate and the interleukins may be applied to the blood cells at the same time. The phosphate may be applied after the interleukin is applied to the blood cells.

Examples of the culture medium in which the blood cells are cultured include, but are not limited to, the RPMI1640 medium, the minimum essential medium (α-MEM), the Dulbecco's modified Eagle medium (DMEM), and the F12 medium.

Next, an inducer is introduced into the blood cells to which at least the phosphate has been applied, whereby stem cells are induced from the blood cells. In inducing the stem cells, inducing refers to reprogramming, initialization, transformation, and cell fate reprogramming.

The inducer introduced into the blood cells may be RNA. The RNA may be mRNA. The inducer includes, for example, OCT3/4, SOX2, KLF4, and c-MYC. M₃O, which is obtained by improving OCT3/4, may be used as an inducer. In addition, the inducers may be at least one selected from the group consisting of LIN28A, FOXH1, LIN28B, GLIS1, p53-dominant negative, p53-P275S, L-MYC, NANOG, DPPA2, DPPA4, DPPA5, ZIC3, BCL-2, E-RAS, TPT1, SALL2, NAC1, DAX1, TERT, ZNF 206, FOXD3, REX1, UTF1, KLF2, KLF5, ESRRB, miR-291-3p, miR-294, miR-295, NR5A1, NR5A2, TBX3, MBD3sh, TH2A, TH2B, and P53DD. The RNA of each of these inducers is available from TriLink BioTechnologies. It is noted that although the genetic symbols are denoted in terms of the human, the species is not intended to be restricted by upper or lower case letter notation. For example, even in a case where only upper case letter notation is applied to a gene, it does not exclude that a mouse or rat gene is included. However, in Examples, the genetic symbols are denoted according to the actually used biological species.

The RNA of the inducer may be modified with at least one selected from the group consisting of pseudouridine (Ψ), 5-methyl uridine (5meU), N1-methyl pseudouridine (me1Ψ), 5-methoxy uridine (5moU), 5-hydroxymethyl uridine (5hmU), 5-formyl uridine (5fU), 5-carboxymethyl ester uridine (5camU), thienoguanosine (thG), N4-methyl cytidine (me4C), 5-methyl cytidine (m5C), 5-methoxycytidine (5moC), 5-hydroxymethyl cytidine (5hmC), 5-hydroxycytidine (5hoC), 5-formcytidine (5fC), 5-carboxycytidine (5caC), N6-methyl-2-aminoadenosine (m6DAP), diaminopurine (DAP), 5-methyl uridine (m5U), 2′-O-methyl uridine (Um or m2′-OU), 2-thiouridine (s20), and N6-methyl adenosine (m6A).

The RNA of the inducer may be polyadenylated.

The RNA of the inducer may be prepared by polyadenylation of an RNA which is obtained by in vitro transcription (IVT). The RNA may be polyadenylated during the IVT by using a DNA template that encodes a poly (A) terminal. The RNA may be capped. In order to maximize the efficiency of expression in cells, it is preferable that most RNA molecules preferably contain a cap. The RNA may have a 5′ cap [m7G (5′)ppp(5′)G] structure. The sequence is a sequence that stabilizes an RNA and promotes transcription. 5′ triphosphate may be removed from an RNA having a 5′ triphosphate by a dephosphorylation treatment. The RNA may have [3′0-Me-m7G(5′)ppp(5′)G] as an Anti-Reverse Cap Analog (ARCA). The ARCA is a sequence to be inserted before the transcription start site, and the efficiency of the RNA to be transcribed is doubled. The RNA may have a Poly A tail.

In addition, the RNA of the inducer may be a replicative RNA having a self-proliferative ability. The replicative RNA is an RNA having a self-proliferative ability, and unlike a normal RNA, it also has the ability to express a protein required for RNA replication. The replicative RNA is derived from the Venezuelan equine encephalitis (VEE) virus, which is one kind of alpha virus. In a case where a replicative RNA is transfected into cells, it is possible to express an RNA that continues to produce an inducer, and thus it is possible to eliminate a plurality of times of the introduction of the RNA into the cells.

The sequence of the replicative RNA may include a sequence obtained from α virus selected from the group consisting of an alpha virus replicon RNA, eastern equine encephalitis virus (EEE), Venezuelan equine encephalitis virus (VEE), Everglades virus, Mucambo virus, Pixuna virus, and western equine encephalitis virus (WEE).

In addition, the replicative RNA may include a sequence obtained from a virus selected from the group consisting of Sindbis virus, Semliki Forest virus, Middelburg virus, Chikungunya virus, O'nyong-nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus, and Buggy Creek virus.

The replicative RNA contains, for example, (VEE RNA replicase)-(promoter)-(RF1)-(self-cleavable peptide)-(RF2)-(self-cleavable peptide)-(RF3)-(IRES or core promoter)-(RF4)-(IRES or any promoter)-(any selectable marker)-(VEE 3′UTR and poly-A tail)-(any selectable marker)-promoter, from 5′ toward 3′. The above-described RF1-4 is a factor that induces dedifferentiation of somatic cells into pluripotent cells. The above-described RF2-3, RF3-4, and RF4 are arbitrary. The above-described RF1-4 may be selected from the group consisting of OCT3/4, KLF4, SOX-2, c-MYC, LIN28A, LIN28B, GLIS1, FOXH1, p53-dominant negative, p53-P275S, L-MYC, NANOG, DPPA2, DPPA4, DPPA5, ZIC3, BCL-2, E-RAS, TPT1, SALL2, NAC1, DAX1, TERT, ZNF 206, FOXD3, REX1, UTF1, KLF2, KLF5, ESRRB, miR-291-3p, miR-294, miR-295, NR5A1, NR5A2, TBX3, MBD3sh, TH2A, and TH2B.

Any method for introducing an inducer into blood cells may be used. For example, an inducer may be introduced into blood cells by using a vector. An inducer may be introduced into blood cells by lipofection.

A Sendai virus (Sev) can be used as a vector. The Sendai virus is a virus having an RNA as the genome, which belongs to Mononegavirales, Paramyxoviridae. The Sendai virus has an RNA genome and an envelope consisting of a lipid bilayer encapsulating the RNA.

As the Sendai virus delivering the RNA of the inducer, CytoTune (registered trademark, Invitrogen) can be used. The Sendai virus vector may be a Sendai virus vector having improved persistent infectivity. The Sendai virus vector having improved persistent infectivity is also referred to as a stealth type RNA vector. As the stealth type RNA vector, an SRV iPSC-1 Vector, an SRV iPSC-2 Vector, an SRV iPSC-3 Vector, or an SRV iPSC-4 Vector (registered trademark, TOKIWA-Bio inc.) can be used. Details of the stealth type RNA vector are described in Japanese Patent No. 4478788, Japanese Patent No. 4936482, Japanese Patent No. 5633075, and Japanese Patent No. 5963309.

An index of the titer of the Sendai virus includes multiplicity of infection (MOI). The MOI of the Sendai virus is, for example, 0.1 to 100.0, or 1.0 to 50.0.

The inducer may be introduced into blood cells which have been subjected to adhesion culture, or the inducer may be introduced into blood cells that are subjected to suspension culture in a gel medium.

The blood cells into which the inducer has been introduced may be subject to feeder-free culturing by using a basement membrane matrix such as Matrigel (Corning Inc.), CELLstart (registered trademark, Thermo Fisher Scientific, Inc.), or Laminin 511 (iMatrix-511, Nippi, Incorporated).

As a culture medium in which the blood cells into which the inducer has been introduced are cultured, it is possible to use, for example, a stem cell culture medium such as a human ES/iPS medium such as Stemfit (Ajinomoto Co., Inc.).

However, the stem cell culture medium is not limited to this, and various stem cell culture media can be used. For example, Primate ES Cell Medium, mTeSR1, TeSR2 (STEMCELL Technologies Inc.), or the like may be used. The stem cell culture medium is put into, for example, a plate, a well, a tube, or the like.

The gel medium does not contain a growth factor such as basic fibroblast growth factor (bFGF). Alternatively, the gel medium contains a growth factor such as bFGF at a low concentration of 400 μg/L or less, 40 μg/L or less, or 10 μg/L or less.

In addition, the gel medium does not contain TGF-β or contains TGF-β at a low concentration of 600 ng/L or less, 300 ng/L or less, or 100 ng/L or less.

The gel medium does not have to be stirred. In addition, the gel medium may not contain feeder cells.

The gel medium may contain at least one substance selected from the group consisting of cadherin, laminin, fibronectin, and vitronectin.

After the introduction of an inducer into blood cells, the cells may be subjected to initialization in a liquid culture medium other than the gel medium, or the cells may be subjected to initialization in the gel medium.

After introducing the inducer into the blood cells and then culturing the cells, a passage including recovering the cells into which the inducer has been introduced and seeding, in the culture medium, at least a part of the cells that have been mixed by recovering may be subjected to at least one time. In the passage, clones of the cells into which the inducer has been introduced may be mixed with each other. In the passage, different clones of the cells into which the inducer has been introduced may be mixed with each other. Then, an operation of recovering the cells into which the inducer has been introduced, and seeding, in a culture medium, at least a part of the cells that have been mixed by recovering may be carried out a plurality of times. Until stem cells are established, the cells into which the inducer has been introduced are recovered, and at least a part of the recovered cells that have been mixed may be seeded in a culture medium to carry out the passage. It is noted that all of the recovered cells that have been mixed may be seeded in a culture medium.

Here, the passage containing recovering the cells into which the inducer has been introduced and seeding, in the culture medium, at least a part of the cells that have been mixed by recovering refers to that, for example, the cells in which the inducer has been introduced are passaged without distinguishment according to the genetic expression state. For example, the cells into which the inducer has been introduced may be seeded in the same culture vessel at the time of passage, without distinguishment according to the genetic expression state. Alternatively, the passage containing recovering the cells into which the inducer has been introduced and seeding, in the culture medium, at least a part of the cells that have been mixed by recovering refers to that, for example, the cells in which the inducer has been introduced are passaged without distinguishment according to the degree of reprogramming. For example, the cells into which the inducer has been introduced may be seeded in the same culture vessel at the time of passage, without distinguishment according to the degree of reprogramming.

Alternatively, the passage containing recovering the cells into which the inducer has been introduced and seeding, in the culture medium, at least a part of the cells that have been mixed by recovering refers to that, for example, the cells in which the inducer has been introduced are passaged without distinguishment according to the morphology. For example, the cells into which the inducer has been introduced may be seeded in the same culture vessel at the time of passage, without distinguishment according to the morphology. Alternatively, the passage containing recovering the cells into which the inducer has been introduced and seeding, in the culture medium, at least a part of the cells that have been mixed by recovering refers to that, for example, the cells in which the inducer has been introduced are passaged without distinguishment according to the size. For example, the cells into which the inducer has been introduced may be seeded in the same culture vessel at the time of passage, without distinguishment according to the size.

In addition, alternatively, the passage containing recovering the cells into which the inducer has been introduced and seeding, in the culture medium, at least a part of the cells that have been mixed by recovering refers to that the cells in which the inducer has been introduced are passaged without cloning. For example, in a case of passaging without cloning, it is not necessary to pick up colonies which are formed by the cells into which the inducer has been introduced. For example, in a case of passaging without cloning, it is not necessary to separate a plurality of colonies from each other, which are formed by the cells into which the inducer has been introduced. For example, cells that have formed a plurality of different colonies may be mixed and seeded in the same culture vessel at the time of passage. In addition, for example, in a case of passaging without cloning, it is not necessary to carry out cloning of single colonies which are formed by the cells into which the inducer has been introduced. For example, colonies may be mixed with each other and seeded in the same culture vessel at the time of passage.

For example, in a case where the cells into which the inducer has been introduced have been subjected to adhesion culture, the cells which have been subjected to the adhesion culture are recovered, and at least a part of the cells that have been mixed by recovering may be seeded in a culture medium to carry out passage. For example, at the time of passage, the cells are detached from the culture vessel, and at least a part of the cells that are mixed by detaching may be seeded in the same culture vessel. For example, the cells may be detached from the culture vessel with a stripping solution, and the entire cells that have been mixed by detaching may be passaged. For example, the cells that have not formed a colony may be passaged. In a case where the cells into which the inducer has been introduced have been subjected to suspension culture, the entire cells which have been subjected to the suspension culture may be passaged.

In a case where the cells into which the inducer has been introduced are passaged, the cells may be seeded in a culture medium or a culture vessel at a low concentration. Here, the low concentration is, for example, 1 cell/cm² or more, and 0.25×10⁴ cells/cm² or less, 1.25×10³ cells/cm² or less, 0.25×10³ cells/cm² or less, 0.25×10² cells/cm² or less, or 0.25×10¹ cells/cm² or less. Alternatively, the low concentration is a concentration at which 10 cells or less, 9 cells or less, 8 cells or less, 7 cells or less, 6 cells or less, 5 cells or less, 4 cells or less, 3 cells or less, or 2 cells or less are capable of coming into contact with each other, and 11 or more cells do not come into contact with each other. It is noted that a plurality of cell clumps, in which 10 or less cells are in contact with each other, may be present. Alternatively, in a case where a state in which the entire bottom surface of the cell container is covered with cells is defined as 100% of confluency, the low concentration is 5% or less of confluency, 4% or less of confluency, 3% or less of confluency, 2% or less of confluency, 1% or less of confluency, 0.5% or less of confluency, 0.1% or less of confluency, 0.05% or less of confluency, or 0.01% or less of confluency. In addition, alternatively, the low concentration is, for example, a concentration at which single cells in the seeded cells do not come into contact with each other. For example, single cells may be seeded in wells of a well plate. The well plate may be a 12-well plate or a 96-well plate. According to the findings of the present inventors, in a case where the cells into which the inducer has been introduced are passaged, it is possible to suppress the residual of the Sendai virus in the stem cells induced from the cells by seeding the cells in the culture medium at the low concentration. In the induced stem cells, the proportion of cells in which the Sendai virus remains is, for example, 4% or less, 3% or less, 2% or less, 1% or less, 0.5% or less, or 0%.

The cells into which the inducer has been introduced may be cultured and passaged in a closed culture vessel. The closed culture vessel does not exchange, for example, gases, viruses, bacteria, impurities, and the like with the outside. In addition, the cells into which the inducer has been introduced may be subjected to expansion culture by two-dimensional culturing or may be subjected to expansion culture by three-dimensional culturing.

After the cells into which an inducer has been introduced are induced into stem cells, and the stem cells are established, the entire cells which have been subjected to adhesion culture may be subjected to cryopreservation as the stem cells. For example, the entire cells detached from the culture vessel with a stripping solution may be subjected to cryopreservation as the stem cells. In addition, after the cells into which an inducer has been introduced are induced into stem cells, the entire cells which have been subjected to suspension culture may be subjected to cryopreservation as the stem cells.

The induced stem cells can express Nanog, OCT4, SOX2, and the like, which are markers for undifferentiated cells. The induced stem cells can express TERT. The induced stem cells can exhibit telomerase activity.

In addition, it is possible to confirm whether or not the stem cells have been induced from the blood cells, for example, from the morphology of the cells. For example, the induced stem cells can form flat colonies similar to ES cells and express an alkaline phosphatase. Alternatively, whether or not the stem cells have been induced from the blood cells may be confirmed by analyzing, with a flow cytometer, whether or not at least one surface marker selected from TRA-1-60, TRA-1-81, SSEA-1, and SSEA-5, which are cell surface markers that indicate undifferentiation, is positive. TRA-1-60 is an iPS/ES cell-specific antigen and thus is not detected in somatic cells. Since iPS cells can be obtained only from a TRA-1-60 positive fraction, the TRA-1-60 positive cells are considered to be iPS cells.

The induced stem cells include, for example, a rearranged γδ-TCR gene. The rearranged γδ-TCR gene is a gene encoding TCR in which the rearrangement of the TCRγ region and the TCRδ region has occurred. The TCRγ region includes Vγ-Jγ. The TCRδ region includes Vδ-Dδ-Jδ. The induced stem cells include, for example, the rearranged γδ-TCR gene having the J1/J2 gene.

A method for producing blood cells according to the present embodiment includes preparing the stem cells produced by the above-described method for producing stem cells and inducing blood cells from the stem cells.

A method for inducing blood cells from stem cells is not particularly limited. For example, the prepared cells are cultured for 4 days in a culture medium containing a GSK3 indicator such as CHIR99021, a bone morphogenetic protein such as BMP-4, and a growth factor such as VEGF. Further, the cells are cultured for 2 days in a culture medium containing an ALK5 indicator such as SB431542, growth factors such as VEGF and bFGF, and a stem cell factor (SCF). Further, the cells are cultured for 2 days in a culture medium containing a growth factor such as VEGF, SCF, interleukins such as IL-3 and IL-6, a cytokine such as F1t3L, and erythropoietin (EPO). Further, the cells are cultured in a culture medium containing SCF, an interleukin such as IL-6, and EPO. As a result, the blood cells are induced.

Alternatively, the stem cells may be seeded on stromal cells (stroma cells) to induce the blood cells from the stem cells. The stromal cells may be derived from the bone marrow. The stromal cell may be an OP9 cell. The OP9 cell does not produce macrophage colony-stimulating factor (M-CSF) but has a function of supporting the differentiation of the stem cells into the blood cells. For example, colonies of the stem cells are divided into a plurality of cell clumps, and the cell clumps of the stem cells are seeded on the OP9 cells as feeder cells. As a result, the blood cells are induced from the stem cells. The induced blood cells are, for example, positive to positive CD34 and CD43.

The induced blood cells may be the γδ-type T cells.

### Example 1

An RPMI (Roswell Park Memorial Institute) 1640 medium (Gibco) containing 10 nmol/L (E)-4-hydroxy-3-methyl-but-2-enyl pyrophosphate (HMBPP, Sigma-Aldrich Co., LLC, registered trademark), 10% fetal bovine serum (Life Technologies), 1.0×10⁻⁵ mol/L 2-mercaptoethanol (Nacalai Tesque, Inc.), 100 U/mL penicillin, and 100 μg/mL streptomycin (Life Technologies) was prepared as an HMBPP-containing medium.

Human peripheral blood mononuclear cells were put into the culture medium, and a culture medium containing about 1×10⁶ monocytes was put into a 24-well plate (1st day). 1 μL of 20 μg/mL IL-2 was added to the culture medium every day. On the third day, the culture medium containing the cells was recovered from the plates, the culture medium was centrifugally separated, and the supernatant was removed. Then, 2 mL of a new HMBPP-containing medium was added to the cells, and 1 mL was put into each of two wells of a 24-well plate.

On the 6th day, the culture medium containing the cells was recovered from the plates, the culture medium was centrifugally separated, and the supernatant was removed. Then, an HMBPP-containing medium was added to the cells, and a culture medium containing 2×10⁴ monocytes was put into a 96-well plate. KLF4, OCT3/4, SOX2, and c-MYC were introduced into the cells by using CytoTune-iPS 2.0 (Thermo Fisher Scientific, Inc.). The multiplicity of infection (MOI) was adjusted to be 20 to 30.

On the 7th day, the culture medium containing the cells recovered from the 96-well plate were added with a fresh HMBPP-containing medium and put into a 6-well plate. On the 8th, 10th, and 12th days, a stem cell culture medium (Stem Fit, Ajinomoto Co., Inc.) was added thereto, and thereafter, the culture medium was replaced using the stem cell culture medium.

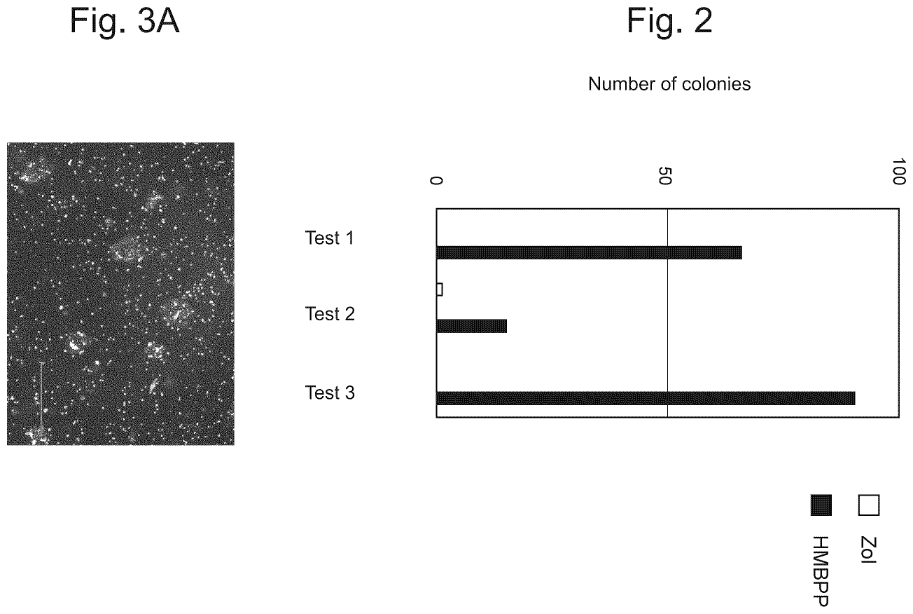

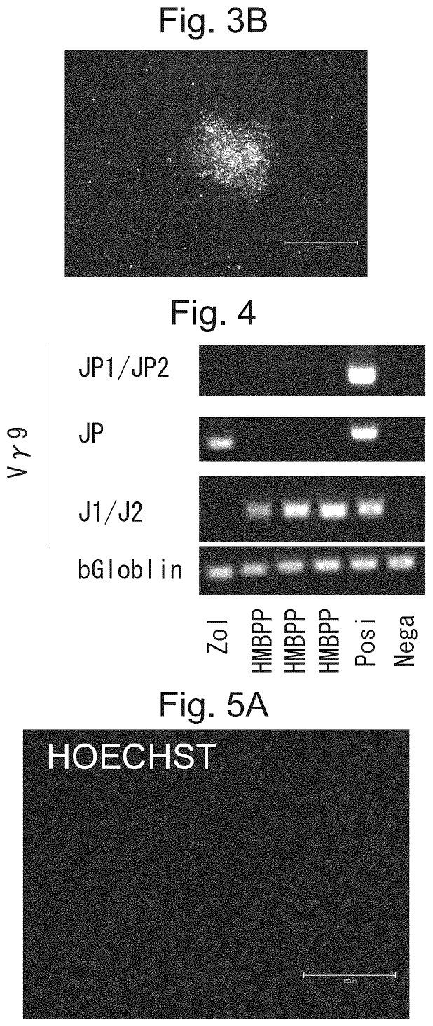

As shown in FIG. 2, it was confirmed that a plurality of colonies of iPS cells has been formed on the 14th day. A photographic image of the established iPS cells is shown in FIG. 3. In addition, genomic DNA was extracted from iPS cells, and it was analyzed by PCR and electrophoresis whether or not the cells had a Vγ9 gene that underwent rearrangement. The genomic DNA of the γδT cells as a positive control and the genome of iPS cells, as a negative control, which did not have a Vγ9 gene that underwent rearrangement, were analyzed in the same manner. As a result, as shown in FIG. 4, it was confirmed that the iPS cells established in Example 1 have a Vγ9 gene that has undergone rearrangement, the Vγ9 gene having the J1/J2 gene.

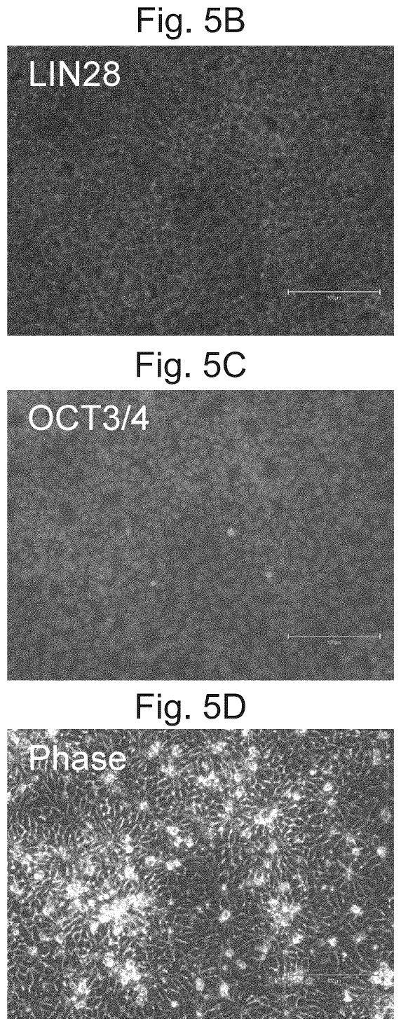

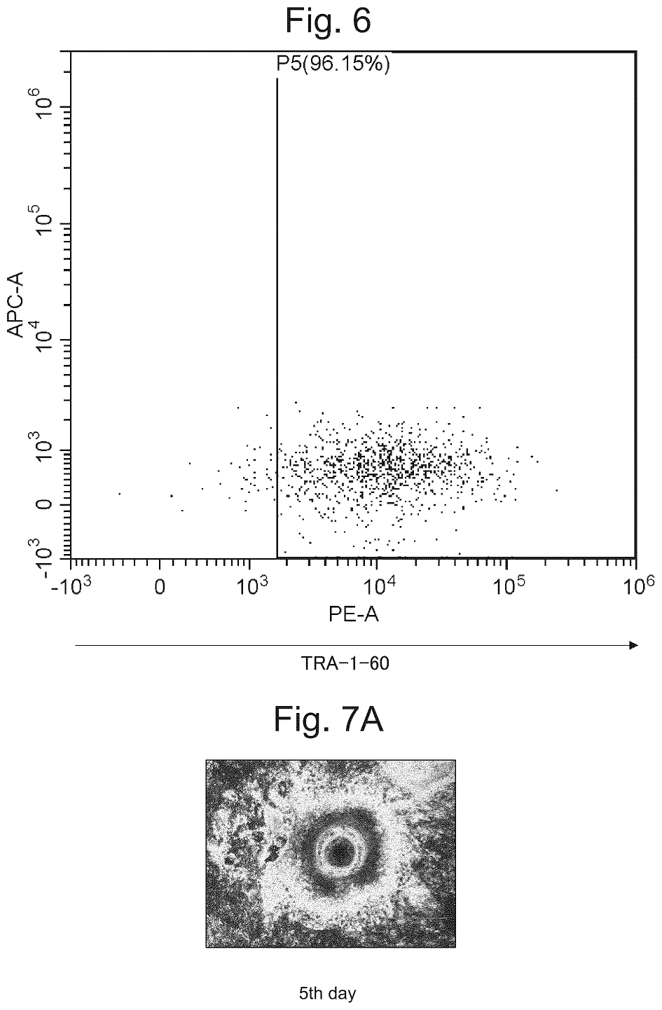

In addition, as a result of immunostaining the established iPS cells with antibodies against LIN28 and OCT3/4, which are markers of pluripotent stem cells, the cells were positive to LIN28 and OCT3/4 as shown in FIG. 5. Further, as a result of analyzing the established iPS cells with a flow cytometer, the cells were positive to TRA-1-60 as shown in FIG. 6.

### Comparative Example 1

iPS cells were induced in the same manner as in Example 1, except that HMBPP in the HMBPP-containing medium was changed to 5 μL of zoledronic acid (Zol, Sigma-Aldrich Co., LLC). As shown in FIG. 2, colonies of the iPS cells were not formed in the tests 1 and 3 of Comparative Example 1. In the test 2 of the comparative example, although colonies of the iPS cells were formed, the number thereof was significantly small as compared with that in the test 2 of Example 1. As shown in FIG. 4, it was confirmed that the iPS cells established in Comparative Example 1 does not have a Vγ9 gene that has undergone rearrangement, the Vγ9 gene having the J1/J2 gene.

### Example 2

The colonies of the iPS cells prepared in Example 1 were detached from the culture vessel with 0.25% trypsin and 1 mg/mL collagenase IV and divided into a plurality of cell clumps by pipetting. Culture vessels, in which OP9 cells and OP9/DLL1 cells were cultured as feeder cells, were prepared. The OP9 cells and the OP9/DLL1 cells were cultured in an α-MEM medium to which 20% fetal bovine serum (FBS) was added. A plurality of cell clumps consisting of the iPS cells was seeded on each kind of the feeder cells.

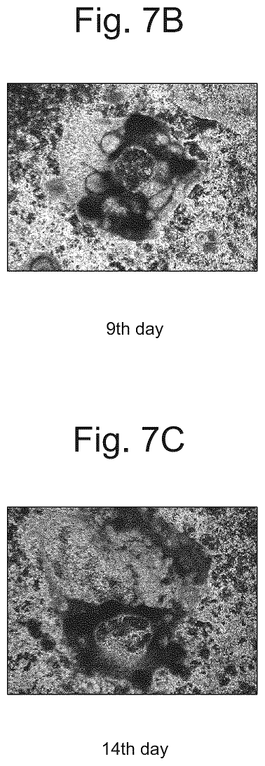

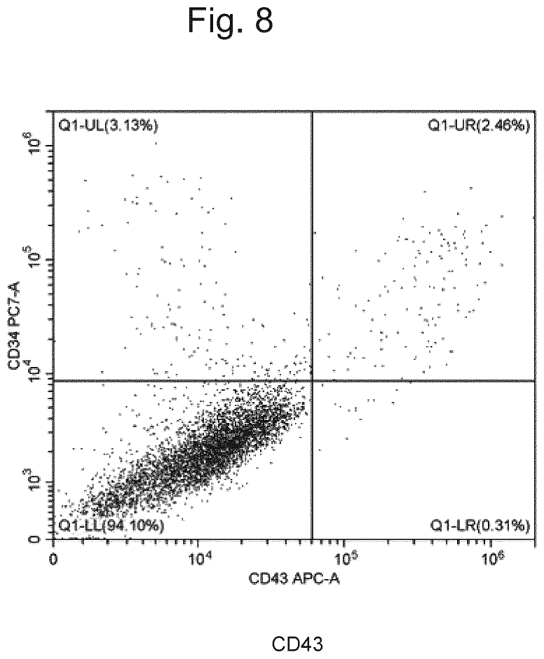

FIG. 7 shows the photographic images of the cells on the 5th, 9th, and 14th days after the iPS cells were seeded on the feeder cells. The process of differentiation of the iPS cells into blood progenitor cells was observed. In addition, FIG. 8 shows the results obtained by analyzing the cells on the 14th day by flow cytometry. It has been confirmed that the cells are positive to CD34 and CD43, which are marker of blood cells.

1. A method for producing stem cells, comprising:
applying a phosphate to blood cells; and
inducing stem cells from the blood cells.

2. The method for producing stem cells according to claim 1, wherein the phosphate is an intermediate product or a final product in a non-mevalonate pathway.

3. The method for producing stem cells according to claim 1, wherein the phosphate is (E)-4-hydroxy-3-methyl-but-2-enyl pyrophosphate.

4. The method for producing stem cells according to claim 1, further comprising applying an interleukin to the blood cells.

5. The method for producing stem cells according to claim 4, wherein the interleukin is at least one selected from the group consisting of IL-2, IL-15, and IL-23.

6. The method for producing stem cells according to claim 1, wherein the blood cells are monocytes.

7. The method for producing stem cells according to claim 1, wherein the stem cells are iPS cells.

8. The method for producing stem cells according to claim 1, wherein in the inducing of the stem cells from the blood cells, an inducer RNA is introduced into the blood cells.

9. The method for producing stem cells according to claim 1, wherein in the inducing of the stem cells from the blood cells, a Sendai virus vector is used.

10. The method for producing stem cells according to claim 1, wherein the stem cells contain rearranged γδ-TCR genes.

11. A method for producing blood cells, comprising:
preparing stem cells produced by the method for producing stem cells according to claim 1; and
inducing blood cells from the stem cells.

12. The method for producing blood cells according to claim 11, wherein the blood cells are γδ-type T cells.

13. The method according to claim 11, wherein in the inducing of the blood cells from the stem cells, a cell clump of the stem cells is seeded on feeder cells.

14. The method according to claim 13, wherein the feeder cells are stromal cells.
